(19) United States
(12) Patent Application Publication
(10) Pub. No.: US 2006/0018950 A1
(43) Pub. Date: Jan. 26, 2006

(54) Body ornamentation

(76) Inventor: Timothy C. Probasco, Fort Lauderdale, FL (US)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 10/896,543

(22) Filed: Jul. 22, 2004

(51) Int. Cl. A61K 9/70

(52) U.S. Cl. 424/443; 2/1

(57) ABSTRACT

A pattern is applied to human skin by applying a patch in the shape of the pattern to the skin. Sun tan lotion or sun tan oil is then applied to the patch and skin. The patch is unitary, flexible, and impervious to penetration by sun tan lotion and sun tan oil as well as by ultra-violet rays. When the patch is removed, a pattern corresponding to the shape of the patch is exposed.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

This invention relates to a system for utilizing ultraviolet rays to form a pattern on human skin, the pattern being formed by skin of a lighter color than skin contiguous thereto. More particularly the invention incorporates a method of forming a predetermined-pattern on human skin and a multi-laminate sheet product for forming a patch for application to human skin to form the predetermined pattern.

### BACKGROUND OF THE INVENTION

U.S. Pat. No. 5,052,418, issued Oct. 1, 1991, discloses an approach for producing a temporary predetermined image on the human skin by the action of skin tanning rays. The approach includes applying a template to human skin. The template has an opening therein which corresponds to the image one wishes to duplicate on the skin. A sun block, such as colored zinc oxide based paste, cream or lotion is applied to the skin through the opening to provide a sun block pattern forming the image. The template is removed and the user's skin will not be exposed to the sun where the sun block is located. Once the skin has been exposed to tanning rays, the sun block is washed away from the skin to reveal a lighter or untanned area of skin.

It will be appreciated that with the system just described, the shape of the untanned skin is dictated by the zinc oxide based paste, cream or lotion forming the design. The problem, however, is that the image formed by the paste, cream or lotion can lose its shape if rubbed or otherwise impacted or if it is fully or partially washed away by perspiration or water. In other words, utilizing the approach of U.S. Pat. No. 5,052,418, it is highly possible that a well-defined evenly tanned (or untanned) area of skin will not be created.

U.S. Pat. No. 1,854,821, issued Apr. 19, 1932, discloses wearing apparel, and particularly bathing suits, wherein a design is created by an opening formed in the apparel. This allegedly results in creation of a pattern on the wearer's body corresponding to the opening in the garment. The difficulty with this approach is that any relative movement between the garment and the wearer's body will adversely affect the pattern formed on the skin. Of course, such movement is highly likely with bathing suits or other sports-attire. Further, such an approach makes it undesirable to use sun tan lotions or sun tan oils since staining of the apparel can occur.

### SUMMARY OF THE INVENTION

The system of the present invention employs ultra-violet rays to form a predetermined pattern on human skin. The invention incorporates both a method and a device. By practicing the teachings of the present invention, a precisely defined pattern will be reproduced on human skin.

The system employs a patch which remains in place on the skin throughout the entire period of pattern replication. The patch employed firmly adheres to the skin and is highly flexible so that it will conform to the skin even during exercise. Furthermore, the pattern will be formed even if sun tan lotion or sun tan oil utilized in practicing the method of the invention is disturbed by touch or diluted by perspiration or other liquid.

The method of the present invention is for forming a predetermined pattern on human skin. The method includes the step of forming a discrete, unitary, flexible patch impervious to penetration by sun tan lotion and sun tan oil and substantially impenetrable by ultra-violet rays and having said predetermined pattern from a sheet of flexible material impervious to penetration by sun tan lotion and sun tan oil and substantially impenetrable by ultra-violet rays.

The patch is secured to the skin of a human body with adhesive at a selected location on the human body after determining that the skin at the selected location is substantially free from foreign matter which would adversely effect adhesion to the skin.

Next, either sun tan lotion or sun tan oil is applied to the skin at the selected location until all skin contiguous to the patch and not covered thereby is coated with the sun tan lotion or sun tan oil.

During the application step, the sun tan lotion or sun tan oil are prevented from engaging that portion of the skin covered by the patch.

The coated skin and patch are exposed to ultra-violet rays for a period of time sufficient to darken the color of the coated skin due to such exposure.

After elapse of the period of time, the patch is removed from the skin to expose the skin portion and display the predetermined pattern on the skin, said skin portion being of a different color than the coated skin.

The present invention also encompasses a multi-laminate sheet product for forming a patch for application to human skin, the patch having a predetermined pattern to be duplicated on the skin by shielding the skin from exposure to ultra-violet rays.

The multi-laminate sheet product includes a layer of flexible material impervious to penetration by sun tan lotion and sun tan oil and substantially impenetrable by ultra-violet rays.

A layer of adhesive is bonded to the layer of flexible material for adhesive securement to human skin to releasably secure the layer of flexible material to the skin. The layer of flexible material and the layer of adhesive are cut in the predetermined pattern and the adhesive secures the flexible material to the skin over the complete pattern.

A protector sheet is releasably attached to the adhesive in opposition to the layer of flexible material, the adhesive being sandwiched between the layer of flexible material and the protector sheet to protect the adhesive from the ambient atmosphere until removal of the protector sheet from the adhesive.

Other features, advantages, and objects of the present invention will become apparent with reference to the following description and accompanying drawings.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 an exploded, perspective view of a multi- laminate sheet product for practicing the teachings of the present invention;

FIG. 2 is a plan view of the sheet product illustrating a patch die cut therein;

FIG. 3 is a plan view of the patch removed from the remainder of the sheet product;

FIG. 4 is an illustration of the patch applied to a human arm;

FIG. 5 is a somewhat diagrammatic view illustrating the patch on a human arm and sun tan lotion being applied to the arm; and

FIG. 6 illustrates the condition of the arm after the patch has been removed therefrom and after the arm has been exposed to ultra-violet rays.

### DETAILED DESCRIPTION OF THE INVENTION

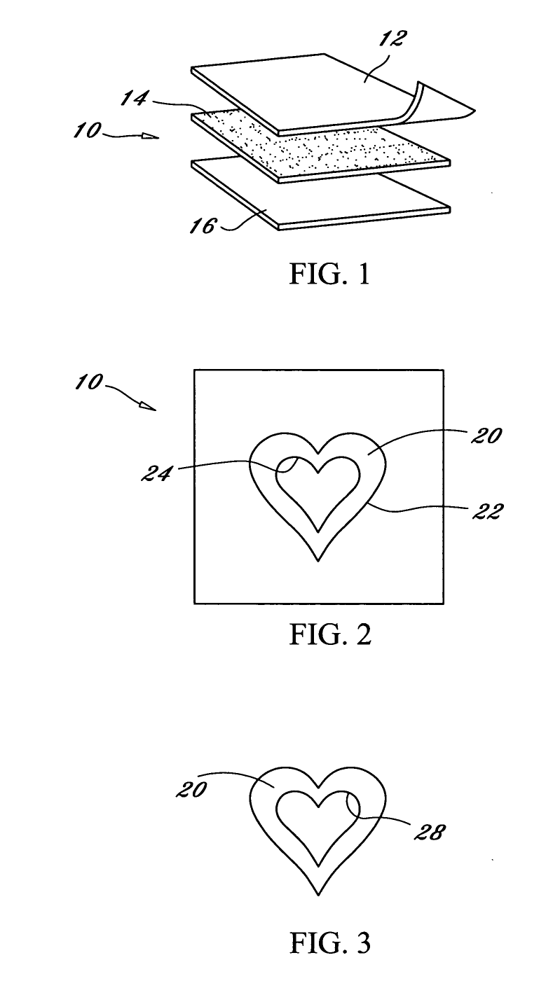

FIG. 1 illustrates three components of a multi-laminate sheet product 10 suitable for forming a patch employed in accordance with the present invention. The sheet product 10 includes a layer 12 of flexible material impervious to penetration by sun tan lotion and sun tan oil and substantially impenetrable by ultra-violet rays. More particularly, the preferred flexible material is plastic sheet material, and even more particularly polyvinylchloride foam tape.

A layer 14 of adhesive is coated or bonded to layer 12, the adhesive being for adhesive securement to human skin to releasably secure the layer of flexible material to the skin. A suitable adhesive is acrylate, designed for medical/surgical applications.

A protector sheet 16 is releasably attached to the adhesive 14 in opposition to the layer 12 of flexible material, the adhesive being sandwiched between the layer of flexible material and the protector sheet to protect the adhesive from the ambient atmosphere until removal of the protector sheet from the adhesive at the time of use.

When practicing the teachings of the present invention, a patch 20 having a predetermined pattern is formed from sheet material 10. In FIG. 2, the patch 20 is in the shape of a heart having an outer periphery 22 established by a line of cut and an inner periphery 24 established by another line of cut. The lines of cut may be formed in any suitable fashion such as by die cutting.

FIG. 3 illustrates the patch removed from the rest of the multi-laminate sheet product. The patch 20 has an inner heart-shaped opening 28. Patch 20 is a discrete, unitary, flexible patch impervious to penetration by sun tan lotion and sun tan oil and substantially impenetrable by ultra-violet rays.

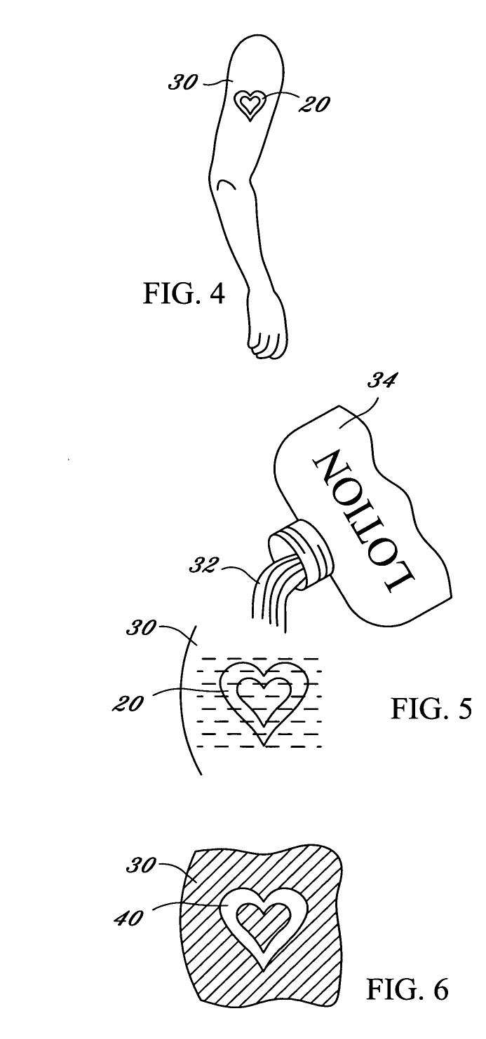

FIG. 4 shows the patch 20 adhesively secured to the skin of a human arm 30. It is, of course, to be understood that the protector sheet 16 has been removed from the patch before application to the arm. Furthermore, the patch should not be applied until it is determined that the patch location, is substantially free from foreign matter which would adversely affect adhesion of the adhesive to the skin.

The next step is to apply either sun tan lotion or sun tan oil to the skin at the location of the patch until all skin contiguous to the patch and not covered thereby is coated with the sun tan lotion or sun tan oil. This step is illustrated in FIG. 5 wherein sun tan lotion 32 is shown being dispensed on the arm from a bottle 34. The sun tan lotion will be worked onto the skin manually and the patch ensures that none of the lotion will penetrate to the skin disposed under the patch.

Next, the coated skin and patch are exposed to ultraviolet rays for a period of time sufficient to darken the color of the coated skin due to such exposure. After exposure to the ultra-violet rays of the sun or other light source has taken place, the patch 20 is removed from the skin to expose the skin previously covered by the patch. FIG. 6 shows the condition of the portion of arm 30 formerly accommodating the patch after the patch has been removed. It will be seen that the portion of the skin formerly covered by the patch is heart-shaped, that is, it is the precise pattern of the patch. The skin portion 40 formerly covered by the patch is a different color, usually lighter, than the contiguous skin which had sun tan oil or sun tan lotion applied thereto.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of forming a predetermined pattern on human skin, said method comprising the steps of:
forming a discrete, unitary, flexible patch impervious to penetration by sun tan lotion and sun tan oil and substantially impenetrable by ultra-violet rays and having said predetermined pattern from a sheet of flexible material impervious to penetration by sun tan lotion and sun tan oil and substantially impenetrable by ultra-violet rays;
adhesively securing said patch to the skin of a human body with adhesive at a selected location on said human body after determining that the skin at said selected location is substantially free from foreign matter which would adversely affect adhesion of said adhesive to said skin;
applying either sun tan lotion or sun tan oil to the skin at said selected location until all skin contiguous to the patch and not covered thereby is coated with the sun tan lotion or sun tan oil;
during said application step, preventing said sun tan lotion or sun tan oil from engaging that portion of the skin covered by said patch;
exposing the coated skin and patch to ultra-violet rays for a period of time sufficient to darken the color of said coated skin due to such exposure; and
after elapse of said period of time, removing said patch from said skin to expose said skin portion and display said predetermined pattern on said skin, said skin portion being of a different color than said coated skin.

2. The method according to claim 1 wherein said sheet of flexible material is a laminate comprised of a layer of plastic and a layer of adhesive.

3. The method according to claim 2 wherein said plastic is polyvinylchloride foam tape.

4. The method according to claim 2 wherein said adhesive is acrylate, designed for medical/surgical applications.

5. The method according to claim 2 including the step of disposing said layer of adhesive between said layer of plastic and a protector sheet and the subsequent step of removing said protector sheet from said layer of adhesive immediately prior to application of said patch to skin.

6. A multi-laminate sheet product for forming a patch for application to human skin, said patch having a predetermined pattern to be duplicated on said skin by shielding said skin from exposure to ultra-violet rays, said multi-laminate sheet product comprising, in combination:
a layer of flexible material impervious to penetration by sun tan lotion and sun tan oil and substantially impenetrable by ultra-violet rays;
a layer of adhesive bonded to said layer of flexible material, said adhesive for adhesive securement to human skin to releasably secure said layer of flexible material to the skin, said layer of flexible material and said layer of adhesive being cut in said predetermined pattern and said adhesive securing the flexible material to the skin over the complete pattern; and
a protector sheet releasably attached to said adhesive in opposition to said layer of flexible material, said adhesive being sandwiched between said layer of flexible material and said protector sheet to protect the adhesive from the ambient atmosphere until removal of said protector sheet from said adhesive.

7. The product according to claim 6 wherein said flexible material is plastic sheet material.

8. The product according to claim 7 wherein said plastic sheet material is polyvinylchloride foam tape.

9. The product according to claim 7 wherein said adhesive is acrylate, designed for medical/surgical applications.
